(19) United States
(12) Patent Application Publication
(10) Pub. No.: US 2019/0388543 A1
(43) Pub. Date: Dec. 26, 2019

(54) FORMULATIONS AND METHODS FOR COMPOSITIONS USED IN WEARABLE DRUG DELIVERY DEVICES

(71) Applicant: Everhart, Inc., Farmington, UT (US)

(72) Inventor: Deborah Dixon, Eureka, UT (US)

(21) Appl. No.: 16/444,668

(22) Filed: Jun. 18, 2019

Related U.S. Application Data
(60) Provisional application No. 62/688,060, filed on Jun. 21, 2018.

(51) Int. Cl. A61K 47/02; A61K 31/122; A61K 36/82; A61K 47/36; A61K 9/70; A61K 47/10; A61K 31/714; A61K 31/522; A61K 36/41; A61K 36/185; A61K 36/77; A61K 36/81

(52) U.S. Cl. CPC A61K 47/02; A61K 31/122; A61K 36/82; A61K 47/36; A61K 9/7061; A61K 36/81; A61K 31/714; A61K 31/522; A61K 36/41; A61K 36/185; A61K 36/77; A61K 47/10

(57) ABSTRACT

Formulations for topical compositions which may be used to deliver drugs. The formulations may be used with a wearable drug delivery device for transdermal delivery. The composition may comprise a clay carrier such as kaolin and/or halloysite, loaded with one or more target compositions. The composition may comprise about 5 to about 15 percent by weight kaolin; about 5 to about 15 percent by weight oil carrier; about 2 to about 3 percent by weight essential oil; about 6.5 to about 26 percent by weight emollient; about 15 to about 20 percent by weight aloe vera barbadensis juice gel; about 0.9 percent to about 5 percent by weight thickening agent; about 0.1 percent to about 5 percent by weight stabilizer; about 2 percent to about 8 percent by weight surfactant; about less than 1 percent by weight target compound; about 0.35 to about 1.8 percent by weight preservative; and about 1 percent to about 5 percent by weight water.

### TECHNICAL FIELD

Described are formulations for topical compositions which may be used to deliver drugs. In particular, the formulations may be used with a wearable drug delivery device for transdermal delivery.

### BACKGROUND

There are many situations in which it is desirable to have a wearable, refillable transdermal drug delivery system and method for use. Single-use patches may also be desirable. Conventional transdermal drug delivery is popular, particularly for use with vitamins, supplements, herbs, and the like.

Transdermal drug delivery has made an important contribution to medical practice. Transdermal delivery systems have continued their steady increase in clinical use for delivery of small, lipophilic, low-dose drugs. Transdermal delivery has a variety of advantages compared with the oral route. In particular, it is used when there is a significant first-pass effect of the liver that can prematurely metabolize drugs. Transdermal delivery also has advantages over hypodermic injections, which are painful, generate dangerous medical waste and pose the risk of disease transmission by needle re-use. In addition, transdermal systems are non-invasive and can be self-administered. They can provide release for long periods of time (up to one week). One example of a transdermal delivery system is shown in U.S. provisional application No. 62/562,671, which is incorporated herein in its entirety by reference.

However, some issues exist with the formulation of compounds to be used in transdermal drug delivery devices. For example, achieving the appropriate viscosity and consistency for transdermal delivery may be difficult. It may be desirable for a transdermal gel which is viscous/thick enough to not spill easily, yet fluid enough to be absorbed by the skin. At the same time, it may be desirable to form such a transdermal gel using as many natural products/ingredients as possible with only a low percentage of synthetic chemicals. It may be difficult to formulate a gel having hydrophilic compounds and lypophilic compounds present in the emulsion/gel, which also have known and proven safety profiles. Similarly, it may be difficult to achieve the desired viscosity, consistency, etc., only using compounds which are known to be safe for transdermal delivery. It may be desirable to optimize the doses of each active ingredient because transdermal dosages may widely differ from those that would be required for oral administration.

Thus, there is a need for a formulation for a compound that may be used to deliver drugs using a wearable, refillable transdermal drug delivery system.

### SUMMARY

Described herein is a method for forming a composition for transdermal drug delivery. The method may comprise the steps of: (1) loading a clay carrier (such as a nanotubular clay carrier, i.e., halloysite, kaolin, etc.) with water soluble active ingredients to form a clay carrier slurry carrying water soluble active ingredients; (2) loading a clay carrier with oil soluble active ingredients to form a clay carrier slurry carrying oil soluble active ingredients; (3) mixing hydrophilic non-active components and adding the clay carrier slurry carrying water soluble active ingredients to form a hydrophilic component; (4) mixing the lipophilic non-active components and adding the clay carrier slurry carrying oil soluble active ingredients to form a lypophilic component; (5) adding the lypophilic component and the hydrophilic component together, and adding at least one thickening agent and at least one preservative.

According to another aspect, disclosed is a composition for use in a transdermal drug delivery device. The composition may comprise: about 5 to about 15 percent by weight clay carrier (i.e., nanotubular kaolin/halloysite); about 5 to about 15 percent by weight oil carrier; about 2 to about 3 percent by weight essential oil; about 6.5 to about 26 percent by weight emollient; about 15 to about 20 percent by weight aloe vera barbadensis juice gel; about 0.9 percent to about 5 percent by weight thickening agent; about 0.1 percent to about 5 percent by weight stabilizer; about 2 percent to about 8 percent by weight surfactant; about less than 1 percent by weight target compound; about 0.35 to about 1.8 percent by weight preservative; and about 1 percent to about 5 percent by weight water.

According to yet another aspect, a method for using a transdermal drug delivery device to deliver a target compound is disclosed. The method may comprise the steps of: selecting a transdermal drug delivery device; loading the transdermal drug delivery device with a viscous compound, the viscous compound comprising: about 5 to about 15 percent by weight clay carrier (by way of example, and not limitation, kaolin/nanotubular clay); about 5 to about 15 percent by weight oil carrier; about 2 to about 3 percent by weight essential oil; about 6.5 to about 26 percent by weight emollient; about 15 to about 20 percent by weight aloe vera barbadensis juice gel; about 0.9 percent to about 5 percent by weight thickening agent; about 0.1 percent to about 5 percent by weight stabilizer; about 2 percent to about 8 percent by weight surfactant; about less than 1 percent by weight target compound; about 0.35 to about 1.8 percent by weight preservative; and about 1 percent to about 5 percent by weight water.

### IN THE DRAWINGS

FIG. 1 shows scanning electron microscopy of halloysite clay mined at NorthstarClay Mines, LLC (Eureka, Utah).

FIG. 2 shows scanning electron microscopy of a source of halloysite from Sigma Aldrich.

### DETAILED DESCRIPTION

The following provides a detailed description of particular embodiments of the present invention. Reference will now be made to the drawings in which the various elements of the illustrated configurations will be given numerical designations and in which the invention will be discussed so as to enable one skilled in the art to make and use the invention. It is to be understood that the following description is only exemplary of the principles of the present invention, and should not be viewed as narrowing the scope of the claims which follow, which claims define the full scope of the invention.

It will be appreciated that various aspects discussed in one drawing may be present and/or used in conjunction with the embodiment shown in another drawing, and each element shown in multiple drawings may be discussed only once. For example, in some cases, detailed description of well-known items or repeated description of substantially the same configurations may be omitted. The reason is to facilitate the understanding of those skilled in the art by avoiding the following description from being unnecessarily redundant. The accompanying drawings and the following description are provided in order for those skilled in the art to fully understand the present disclosure, and these are not intended to limit the gist disclosed in the scope of claims.

It should be noted that the description merely illustrates the principles of the present subject matter. It will thus be appreciated that those skilled in the art will be able to devise various arrangements that, although not explicitly described herein, embody the principles of the present subject matter and are included within its spirit and scope. Furthermore, all examples recited herein are principally intended expressly to be only for pedagogical purposes to aid the reader in understanding the principles of the invention and the concepts contributed by the inventor(s) to furthering the art, and are to be construed as being without limitation to such specifically recited examples and conditions. Moreover, all statements herein reciting principles, aspects, and embodiments of the invention, as well as specific examples thereof, are intended to encompass equivalents thereof.

Reference in the specification to “one configuration” “one embodiment,” “a configuration” or “an embodiment” means that a particular feature, structure, or characteristic described in connection with the configuration is included in at least one configuration, but is not a requirement that such feature, structure or characteristic be present in any particular configuration unless expressly set forth in the claims as being present. The appearances of the phrase “in one configuration” in various places may not necessarily limit the inclusion of a particular element of the invention to a single configuration, rather the element may be included in other or all configurations discussed herein.

Furthermore, the described features, structures, or characteristics of configurations of the invention may be combined in any suitable manner in one or more configurations. In the following description, numerous specific details are provided, such as examples of products or manufacturing techniques that may be used, to provide a thorough understanding of configurations of the invention. One skilled in the relevant art will recognize, however, that configurations of the invention may be practiced without one or more of the specific details, or with other methods, components, materials, and so forth. In other instances, well-known structures, materials, or operations are not shown or described in detail to avoid obscuring aspects of the invention.

Before the present invention is disclosed and described in detail, it should be understood that the present disclosure is not limited to any particular structures, process steps, or materials discussed or disclosed herein, but is extended to include equivalents thereof as would be recognized by those of ordinary skill in the relevant art. More specifically, the invention is defined by the terms set forth in the claims. It should also be understood that terminology contained herein is used for the purpose of describing particular aspects of the invention only and is not intended to limit the invention to the aspects or configurations shown unless expressly indicated as such. Likewise, the discussion of any particular aspect of the invention is not to be understood as a requirement that such aspect is required to be present apart from an express inclusion of the aspect in the claims.

It should also be noted that, as used in this specification and the appended claims, singular forms such as “a,” “an,” and “the” may include the plural unless the context clearly dictates otherwise. Thus, for example, reference to “a water soluble active ingredient” may include one or more of such active ingredients, and reference to “the preservative” may include reference to one or more of such preservatives.

As used herein, the term “substantially” refers to the complete or nearly complete extent or degree of an action, characteristic, property, state, structure, item, or result to function as indicated. For example, a compound, such as a water soluble compound, that is “substantially” solubilized would mean that the compound is either completely solubilized or nearly completely solubilized. The exact allowable degree of deviation from absolute completeness may in some cases depend on the specific context. The use of “substantially” is equally applicable when used in a negative connotation to refer to the complete or near complete lack of an action, characteristic, property, state, structure, item, or result. For example, structure which is “substantially free of” a bottom would either completely lack a bottom or so nearly completely lack a bottom that the effect would be effectively the same as if it lacked a bottom.

As used herein the term “generally” refers to something that is more of the designated adjective than not, or the converse if used in the negative. For example, something maybe said to be generally circular even though it has a somewhat oval shape or is polygonal rather than being completely smooth.

As used herein, the term “about” is used to provide flexibility to a numerical range endpoint by providing that a given value may be “a little above” or “a little below” the endpoint while still accomplishing the function associated with the range.

As used herein, a plurality of items, structural elements, compositional elements, and/or materials may be presented in a common list for convenience. However, these lists should be construed as though each member of the list is individually identified as a separate and unique member.

Concentrations, amounts, proportions and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

In one configuration of this disclosure, a method for loading a clay-carrier compound with compounds to be transdermally delivered is disclosed. A clay-carrier such as halloysite may allow an improved time release of active compounds to be delivered compared to conventional transdermal patches. The nanotubular clay-carrier formulation may also provide better stability of the active compounds, as well as increase bioavailability. The clay nanotubes may penetrate the skin and carry the active compounds as well as oils included in the formulation to facilitate some delivery.

According to one aspect, a composition to be applied to the skin of the user and to deliver compounds transdermally (such as energy enhancing compounds like stimulants) may comprise: a carrier, such as a clay carrier; an additional carrier such as an oil carrier; an essential oil; at least one emollient; at least one thickening agent; at least one stabilizer; at least one stimulant; at least one surfactant; and at least one preservative. In some configurations, a single compound may provide multiple functionalities (such as a compound which acts as a thickener and a stabilizer, etc.).

In one example, the composition may include a carrier comprised of clay, such as kaolinite, halloysite, etc. According to one aspect, the clay carrier may be in a nanotubular form. Nanoparticles within the clay may be able to absorb compositions for topical delivery. Thus, the clay may act as a carrier. The clay may also act as a thickener and/or dispersant for the composition. The nanotubular clay may also offer a cost effective delivery method compared to other common types of delivery (liposomes, capsules, etc.) which are used in cosmetics and pharmaceuticals.

In some configurations, additional carriers such as an oil carrier may be used. Suitable known oil carriers include, but are not limited to, jojoba oil, almond oil, grapeseed oil, olive oil, coconut oil, fractionated coconut oil, shea butter, cocoa butter, sweet almond oil, sunflower oil, wheat germ oil, etc. Oils may be selected for their viscosity, moisture level, fragrance, etc. The carrier oil may function as a carrier, and as an excipient, moisturizer, etc.

The composition may also comprise one or more essential oils. Essential oils may provide a desired fragrance to the composition, and also may function to deliver small molecules that are known to have particular therapeutic effects. For example, a composition that is desired to provide an energizing effect may include essential oils such as citrus oils (orange, lime, grapefruit, etc.), peppermint, eucalyptus, cedarwood, rosemary, thyme, basil, lemongrass, cinnamon, ginger, lemon, spearmint, juniper, frankincense, black pepper, pine, tangerine, lemon peel, etc. A single type of essential oil may be used in the composition, or a blend of essential oils may be used. The essential oil(s) may be selected for their fragrance and/or their known therapeutic effects.

The composition may comprise, as needed or desired, one or more emollients. For example, octyldodecanol may be used, which may serve as an emollient, solvent, and/or moisturizer for the composition. Isopropyl myristate may be used, which may serve as an emollient, and/or moisturizer. Isohexadecane may be used as an emollient and/or skin conditioner. Glycerin may be used as an emollient, humectant, solvent, and/or lubricant. Other known emollients in the art, suitable for contact with skin, may also be used in the composition as desired. In some configurations, the emollient(s) may comprise about five percent by weight to about 25 percent by weight of the composition.

The composition may comprise, as needed or desired, one or more thickening agents. The composition may, in some configurations, have a consistency or thickness that may allow the composition to be used in a wearable, re-fillable as well as a single use drug delivery device. For example, a consistency which is too thin may allow the composition to flow out of the device before delivery is achieved. For example, the formulation may have a viscosity between 10 and 50,000 centipoises. In some configurations, the consistency of the formulation becomes less viscous when it comes in contact with skin of the user.

Suitable thickening agents may include, but are not limited to, aloe vera barbadensis juice gel, carbomer, sodium acrylate/sodium acryloyldimethyl taurate, Hydroxyethylcellulose, and/or hydroxypropyl methylcellulose, etc. Aloe vera barbadensis juice gel as used in the composition may also serve as a moisturizer, thickener, and/or humectant. Carbomer may also serve to stabilize emulsions. Sodium acrylate/sodium acryloyldimethyltaurate copolymer may also serve in gel formation. Hydroxyethylcellulose and/or hydroxypropyl methylcellulose may also serve, in addition as thickeners, to stabilize emulsions. Other thickeners that widely used in the food industry for thickening liquids include xanthum gum, guar gum and gum Arabic. Any other know, suitable thickening agents may be used.

The composition may comprise, as needed or desired, one or more stabilizers. For example, carbomer, triethanolamine, hydroxyethylcellulose and/or hydroxypropyl methylcellulose may be used as stabilizer(s).

The composition may comprise, as needed or desired, one or more surfactants. For example, polyethylene glycol ether of lauryl alcohol (Laureth-3) may be used as a surfactant (and also may serve as an emulsifier and/or dispersant). Similarly, sorbitol, and/or ethylene oxide and oleic acid (polyoxyethylene-20 sorbitan monooleate) (Polysorbate 80) may be used as surfactant(s) (and may also serve as emulsifiers and/or dispersants).

The composition may comprise, as needed or desired, one or more target compounds. In one configuration, for a composition that is desired to have a stimulating effect, the target compound may comprise one or more stimulants or compounds known to provide energy. For example, one or more of the following may be used: vitamin B12(cyanocabalamin), coenzyme Q10 (CoQ10); ashwaghanda root; rhodolia root; caffeine; yerba mate; and/or guarana. It will be appreciated that other target compounds may also be used.

The composition may comprise, as needed or desired, one or more preservatives. For example, benzyl alcohol DHA (dihydroacetic acid), sorbic acid and its salt potassium sorbate, and/or phenoxyethanol, etc. may serve as suitable preservatives. To increase antimicrobial activity more than one preservative can be used for broad range activity again bacteria, yeast and fungi. EDTA Tetrasodium EDTA (ethylenediaminetetraacetic acid tetrasodium salt) is a chelating agent that can also help provide increased preservation when used with other antimicrobial agents.

Table I lists a formulation with ranges for a composition which may be used in a wearable transdermal delivery device. The target compounds include those which may provide an increase in energy.

TABLE I

| | Percentage/Range/Dosage |
|---|---|
| Ingredient/Chemical | (by weight) |
| Kaolin/Halloysite | 5-15 |
| Carrier oil(s) | 5-15 |
| Essential Oil(s) | 2-3 |
| Octyldodecanol | 2-10 |
| Isopropyl myristate | 2-10 |
| Aloe vera barbadensis juice gel | 15-20 |
| Carbomer | 0.1-0.5 |
| Polyethylene glycol ether of lauryl | 1-5 |
| alcohol (Laureth -3) | |
| Sodium acrylate/Sodium | 0.1-0.5 |
| Acryloyldimethyltaurate copolymer | |
| Isohexadecane | 0.5-2 |
| Glycerin | 2-5.5 |
| Sorbitol, ethylene oxide & oleic acid | 1-3 |
| (polyoxyethylene-20 | |
| sorbitanmonooleate) (Polysorbate 80) | |
| Hydroxyethylcellulose | 0.5-3 |
| Hydroxypropyl methylcellulose | 0.2-1 |
| Vitamin B12 (cyanocabalamin) | 1000 ug (<1) |
| CoQ10 | 100 mg (<1) |
| Ashwaghanda root | 100 mg (<1) |
| Rhodolia root | 140 mg (<1) |
| Caffeine | 200 mg (<1) |
| Yerba mate | 85 mg (<1) |
| Guarana | 280 mg (<1) |
| Benzyl alcohol DHA | 0.2-0.8 |
| Sorbic acid and its salt potassium sorbate | 0.15-0.3 |
| Phenoxyethanol | 0.75-1.5 |
| EDTA | 0.1-0.5 |
| Water | 1-15 |

Table II shows an exemplary formulation for a specific formula of a composition to be used in a wearable transdermal drug delivery device. The composition may include target compounds known to improve energy.

TABLE II

| | Percentage/Range/Dosage |
|---|---|
| Ingredient/Chemical | (by weight) |
| Halloysite clay | In slurries (see below, |
| | total of about 15 in slurries) |
| Aloe vera juice | 25 |
| Jojoba oil | 15 |
| Octyldodecanol | 9 |
| Orange oil | 3 |
| Laureth- 3 | 3 |
| Hydroxyethyl cellulose | 3 |
| Sodium acrylate/sodium | 2 |
| acryloyldimethyltaurate copolymer | |
| Isohexadecane | 2 |
| Polysorbate 80 | 1 |
| Phenoxyethanol | 1.5 |
| Carbomer | 0.5 |
| Vitamin B12 slurry | 5 |
| CoQ10 slurry (clay, oil, CoQ10) | 5 |
| Ashwaghanda root slurry (clay, | 5 |
| water, ashwaghanda root) | |
| Rhodolia root slurry (clay, | 5 |
| water, Rhodolia root) | |
| Caffeine slurry (clay, water, | 5 |
| caffeine) | |
| Yerba mate slurry (clay, water, | 5 |
| yerba mate) | |
| Guarana slurry (clay, water | 5 |
| guarana) | |

For each ingredient listed as a “slurry” in table II, the slurry active ingredient, halloysite clay and water. Excepting for the CoQ10 slurry, as CoQ10 is not water soluble. The CoQ10 slurry comprises CoQ10, halloysite clay and carrier oil (jojoba oil).

Table III shows an exemplary formulation for a specific formula of a composition to be used in a wearable transdermal drug delivery device. The composition may include target compounds known to decrease tension/promote calm.

TABLE III

| Ingredient | Concentration/Percentage | |
|---|---|---|
| Halloysite clay | 15 | |
| Aloe vera juice | 25 | |
| Jojoba oil | 15 | |
| Octyldodecanol | 9 | |
| Orange oil | 3 | |
| Laureth- 3 | 3 | |
| Hydroxyethyl cellulose | 3 | |
| Sodium acrylate/sodium | 2 | |
| acryloyldimethyltaurate copolymer | | |
| Isohexadecane | 2 | |
| Polysorbate 80 | 1 | |
| Phenoxyethanol | 1.5 | |
| Carbomer | 0.5 | |
| Chamomile | 220 | mg <1 |
| St John's Wort | 250 | mg <1 |
| Passion Flower | 80 | mg <1 |
| Lemon Balm | 80 | mg <1 |
| Valerian Root | 160 | mg <1 |
| Kava kava | 70 | mg <1 |
| Geranium, Lavender, Bergamot, | 3 | |
| Lemongrass, Patchouli, Ylang Ylang | | |
| Thiamine (B1) | 1.1 | mg <1 |
| Riboflavin (B2) | 1.1 | mg <1 |
| Niacin (B3) | 14 | mg <1 |
| Pantothenic acid (B5) | 5 | mg <1 |
| Pyroxidine (B6) | 1.3 | mg <1 |
| Biotin (B7) | 30 | ug <1 |
| Folic acid (B9) | 250 | mg <1 |
| Cobalamins (B12) | 2.4 | ug <1 |
| 5-Hydroxytryptophan (5-HTP) | 100 | mg <1 |

| | |
|---|---|
| Water | Remainder (~15) |

In some configurations, it may be desirable to form an adhesive patch suitable for wearing against a user's skin. In this case, to achieve the desired adhesion, it may be desirable to add one or more adhesives to the formulation. For example, Loctite Duro-Tak129NA is a self-curing solvent acrylic pressure-sensitive adhesive designed for skin contact where high tack and peel adhesion are required. Other types of adhesives known in the art suitable for dermal contact may be used, such as other Loctite type adhesives or those made by other companies. Table IV shows an exemplary formulation for a specific formula of a composition to be used in a wearable adhesive patch. The composition may include target compounds known to increase energy.

TABLE IV

| Ingredient | Concentration/Percentage |
|---|---|
| Halloysite clay | 12.5 |
| Green tea | 12.5 |
| Guar Gum | 43.5-44 |
| Preservative (such as phenoxyethanol) | 1% to 1.5% |
| 25% Loctite solvent acrylic adhesive | 30 |

### Methods

The following describes an exemplary method for forming a 100 grams of a compound for transdermally delivery, wherein the target compounds comprise compounds known to increase energy. It will be appreciated that this exemplary method is provided by way of example only and does not limit the scope of the appended claims.

### Method A: Preparation for Delivery Via a Wearable Transdermal Drug Delivery Device

Step 1: Preparation of Loaded Nanotubes/Clay

Step 1A: Prepare each of the water soluble/hydrophilic active ingredients and load into nanotubes of clay (water soluble/hydrophilic active ingredients comprise Vitamin B12, Green Tea, Caffeine, Rhodiola, Yerba Mate, Guarana, Ashwaghanda).

In seven separate 20 mL containers (one for each active ingredient), add 1 gram active ingredient (powdered form) followed by 3 mL distilled water at ambient temperature (22° C.). Stir until ingredient is dissolved. Then add 3 grams halloysite clay. To increase the speed of the process, a small amount of heat (100° C.) may be added. The contents are mixed, each 20 mL container is closed (capped) and allowed to sit at room temperature for at least two hours but no more than 24 hours. During this time, the adsorbent nanotubes will swell and incorporate the liquefied active ingredient. A slurry or paste like consistency will form in each container.

Step 1B: Prepare the oil soluble/lypophilic active ingredient(s)(oil soluble/lypophilic active ingredients in this specific example comprise CoEnzyme 10, or CoQ10).

In a 20 mL container, add one gram of CoQ10 and 3 mL almond oil at ambient temperature (22° C.)(jojoba oil may also be used). Next, 3 grams of halloysite clay is added to the container. The contents are stirred and a small amount of heat (just to boil at 100° C.) is applied to cause the nanotubes to expand and incorporate the lypophilic ingredient(s). The slurry is then allowed to sit at room temperature for at least 2 hours but no more than 24 hours. During this time, the adsorbent nanotubes will swell and incorporate the lypophilic active ingredient in the oil slurry.

Step 2: Preparation of Hydrophilic Components.

In a 100 mL container/vessel, while light mixing, add 25 mL aloe vera juice, followed by 5 gm of each of the active ingredient slurries as prepared in Step 1A. Mix thoroughly and add 3 mL of hydroxyethyl cellulose. Continue mixing and add 0.5 grams of carbomer, followed by 3 mL Laureth-3. Mix and set aside until the lypophilic components are ready.

Step 3: Preparation of the Lypophilic Components.

In a 50 mL container/vessel, with light mixing, add 12.5 mL carrier oil (i.e. jojoba), 3 mL essential oil (i.e. orange) and 9 mL octyldodecanol. Mix thoroughly at room temperature. Next add 2 mL isohexadecane. Mix thoroughly and add 5 grams of the lypophilic active ingredient(s) as prepared in Step 1B. Continue mixing and add half the required volume of Sodium acrylate/Sodium Acryloyldimethyltaurate copolymer (1.0 mL). Mix thoroughly.

Step 4: Preparation of Final Gel Formulation

In the 100 mL container holding the hydrophilic components, slowly add the lypophilic components prepared in Step 3, with constant stirring. Add the remaining half of the Sodium acrylate/Sodium Acryloyldimethyl taurate copolymer (1.0 mL) to form the desired gel consistency. Finally add the preservative(s) (1.5 mL of phenoxyethanol) and mix the final formulation to ensure adequate dispersion of the preservative. The final gel formulation may be stored at room temperature.

### Method B: Preparation for Delivery Via an Adhesive Patch

Step 1: Preparation of Loaded Nanotubes/Clay. The active ingredient (for example, green tea) is dissolved in boiling water (the ratio being 1 gram active ingredient:2 mL water for dissolving). While mixing, the dissolved active ingredient is added to the clay in a 1:1 ratio (i.e., 1 gram green tea:1 gram halloysite). The slurry of active ingredient and clay may then be incubated for 2-24 hours at room temperature. It may then be dried at 200° F. to remove or reduce water/moisture for approximately 1-2 hours for 100 grams. It is crushed using a mortar and pestle to form a fine powder.

Step 2: Preparation of Adhesive Components

First, a 3% w/v concentration of guar gum is prepared by dissolving pure guar gum powder in water. This guar gum preparation forms a gel like paste with viscosity of 10-20,000 cP (room temperature). About 1-2 grams of a preservative (such as phenoxyethanol and/or potassium sorbate) is added to the guar gum preparation.

Step 3: Preparation of Final Adhesive Patch

The final adhesive patch may be prepared by loading the patch with the green-tea/halloysite powder, guar gum, and preservative formulation and then dispensing the Loctite adhesive onto the patch. In other configurations, the Loctite adhesive may first be mixed into the formulation (70:30 clay-green tea, guar gum and preservative:adhesive), and then the formulation, including the adhesive, may be loaded onto the patch. The final pH of the formulation is typically around 6.0 (that is, typically between about 5.5 to about 6.5). The appropriate amount of final formulation is dispensed and pressed into the patch material prior to packaging. For example 4-5 g are dispensed per patch described in one application. Depending on the size of the patch and the desired results, more or less of the final formulation may be dispensed onto the patch.

Additional potential formulations are listed in Tables V and VI.

TABLE V

| Ingredient | Percentage |
|---|---|
| Distilled Water | 52.5 |
| Xanthan Gum | 1.1 |
| Vitamin B12-clay slurry | 5.0 |
| Ashwagandha-clay slurry | 5.0 |
| Rhodiola-clay slurry | 5.0 |
| Green Tea-clay slurry | 5.0 |
| Yerba Mate-clay slurry | 5.0 |
| CoQ10-clay slurry | 5.0 |
| Aloe Vera Gel | 2.5 |
| Carbomer | 0.5 |
| Sorbitol | 2.5 |
| Tapioca Starch | 1.4 |
| Jojoba Oil | 4.5 |
| Orange Essential Oil | 0.75 |
| Laureth-3 | 2.25 |
| Halloysite Clay | 0.5 |
| Phenoxyethanol | 1.5 |

To make the formulation described in Table V, the following method may be used: place 1.1 g xanthan gum in 52.5 mL of distilled water and heat to dissolve the xanthan gum. Let it cool and then add the water soluble actives (active-clay slurry). Mix to disperse the slurries into the gum. Combine the Jojoba oil, orange oil and CoQ10-clay slurry and mix well. With mixing add to the gum/water soluble the actives-clay ingredients. Add the aloe vera gel, carbomer and continue to mix the formulation. Next add sorbitol followed by the Laureth-3. Mix the emulsion thoroughly and then add tapioca starch and halloysite clay for final thickening of the gel. Add the preservative and mix thoroughly.

TABLE VI

| Ingredient | Percentage |
|---|---|
| Natural Gel Wax Base (castor seed oil, | 50.0 |
| hydrogenated castor oil, beeswax, carnuba wax) | |
| Jojoba oil | 3.0 |
| Orange Essential oil | 1.0 |
| Sodium Lauryl Sulfate | 3.0 |
| Green Tea-clay slurry | 41.5 |
| Phenoxyethanol | 1.5 |

To make the formulation described in Table VI, the following method may be used: jojoba oil, orange essential oil are added to the natural gel wax base and mixed thoroughly. The Green Tea-clay slurry is added while mixing followed by the Sodium Lauryl Sulfate. Finally, the preservative is added and the formulation is mixed one more time.

Additional characterization of the nanotubular clay was performed to assess the safety and efficacy of the clay as a delivery system. In vitro testing for skin irritation: Non animal toxicity testing was performed on the formulation cited above in Table I and also on Halloysite powder. The goal was to use an assay that was “cruelty free” for predicting skin irritation potential. The EpiDerm™ reconstructed human epidermis model was used for identification and classification of skin irritation hazard according to the United Nations (UN) Globally Harmonized System (GHS). Briefly, toxicity was determined by measuring the relative conversion of MTT (3-(4,5-dimethylthiazol-2-yl)-2.5-diphenyltetrazolium bromide) to a formazan product in the test article treated tissues (Halloysite Powder, Formulation cited in patent) for a sixty minute exposure time, followed by a 42-hour post exposure expression period. The assay protocol used met the requirements of the OECD guideline, “In Vitro Skin Irritation: Reconstructed Human Epidermis Test Method” (TG 439). A test material or article is predicted to be an irritant (GHS Category 1 or 2) if the mean relative viability of the treated tissues is less than or equal to 50% of the mean viability of the negative control. If the mean viability is greater than 50% of the mean viability of the negative control than it is predicted to be a non-irritant.

| In Vitro Result | In Vivo Prediction | GHS Category |
|---|---|---|
| mean tissue viability ≤ 50% | Irritant (I) | Category 1 or 2* |
| mean tissue viability > 50% | Non-irritant (NI) | No Category |

*to discriminate between GHS Category 1 and 2, additional testing required

The Halloysite Powder resulted in 100% mean viability and the formulation shown in Table I had a mean viability of 75% making both the formulation and the raw material Halloysite Powder non-irritants. The high mean relative viability of the treated tissues indicates that the formulation and Halloysite power are both good candidates for patches, since a patch product comes in contact with skin and remains in contact for an extended period of time. The halloysite clay itself was also predicted to be a non-irritant. Characteristics/Properties of the Halloysite Clay Carrier:

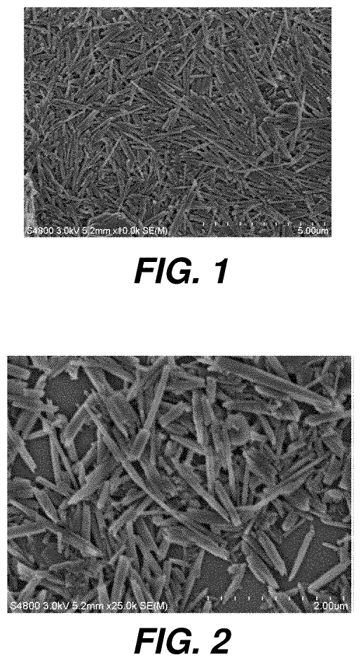

Scanning Electron Microscopy of Halloysite Clay are shown in FIGS. 1 and 2. The data and FIG. 1 are for the halloysite clay mined at NorthstarClay Mines, LLC (Eureka, Utah), and other sources may have different tube measurements. It will be appreciated that these data are listed for example only, and not by way of limitation. Different sources may have different measurements and may be used in conjunction with the present disclosure. For comparison, a source of halloysite from Sigma Aldrich is also given (FIG. 2).p

Zeta potential: −31.9±0.5 mV. No impurities. Very good charge on nanotubes.

Overage length=1200 nm, (70% tubes fall in the interval 600 to 1200 nm). Sigma Aldrich sourced halloysite has length of 1-3 μm (1000-3000 nm). Any nanotubular clay with an approximate length of about 1000 nm to about 3000 nm may be used with the present disclosure.

Overage diameter=140 nm (70% tubes fall in the interval 90 to 180 nm). Sigma Aldrich sourced halloysite has a diameter of 30-70 nm while Northstar's clay diameter is 140 nm. Any clay with a diameter range of about 20 nm to about 150 nm may be used in conjunction with the present disclosure.

Hydrodynamic effective diameter/size: 370±50 nm, indicating a good dispersion of single tubes in water. The properties listed above make halloysite clay a good carrier for transdermal and topical applications. A human skin pore is 50-80 μm in size so the nanotubular clay can easily be adsorbed into the skin.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled.

1. A method for forming a composition for transdermal drug delivery, the method comprising:
loading a clay carrier with water soluble active ingredients to form a clay carrier slurry carrying water soluble active ingredients;
loading a clay carrier with oil soluble active ingredients to form a clay carrier slurry carrying oil soluble active ingredients;
mixing hydrophilic non-active components and adding the clay carrier slurry carrying water soluble active ingredients to form a hydrophilic component;
mixing lipophilic non-active components and adding the clay carrier slurry carrying oil soluble active ingredients to form a lypophilic component;
adding the lypophilic component and the hydrophilic component together, and adding at least one thickening agent and at least one preservative.

2. The method of claim 1, wherein the clay carrier comprises nanotubular halloysite clay.

3. The method of claim 1, wherein the water soluble active ingredients comprise at least one of vitamin B12, green tea, caffeine, rhodiola, yerba mate, guarana, and ashwaghanda.

4. The method of claim 1, wherein the oil soluble active ingredient comprises CoEnzyme 10.

5. The method of claim 1, wherein the hydrophilic non-active components comprise at least one of jojoba oil, almond oil, grapeseed oil, olive oil, coconut oil, fractionated coconut oil, shea butter, cocoa butter, sweet almond oil, sunflower oil, and wheat germ oil.

6. A composition for transdermal delivery, the composition comprising about 10 to about 20 percent by weight clay carrier; about 5 to about 15 percent by weight green tea; about 45 to about 55 percent by weight guar gum; about 1 to about 2 percent by weight preservative; and about 20 to about 30 percent by weight acrylic adhesive.

7. The composition of claim 6, wherein the composition comprises about 15 percent by weight halloysite clay; about 10 percent by weight green tea; about 49 percent by weight guar gum; about 1 percent by weight preservative; and about 25 percent by weight acrylic adhesive.

8. The composition of claim 6, wherein the composition is formed by loading the halloysite clay with green tea solubilized in water to form a slurry; dissolving guar gum powder in water to form a 3% w/v concentration of guar gum; adding preservative; and adding adhesive.

9. The composition of claim 8, wherein the composition is loaded onto a patch for application to skin.

10. The composition of claim 6, wherein the pH of the composition is between 5.0 and 7.0.

11. The composition of claim 6, wherein the preservative comprises at least one of phenoxyethanol, potassium sorbate, and caprylyl glycol.

12. The composition of claim 6, wherein the acrylic adhesive comprises Loctite Duro-Tak129NA.

13. The composition of claim 7, wherein the preservative comprises phenoxyethanol.

14. The composition of claim 7, wherein the acrylic adhesive comprises Loctite Duro-Tak129NA.

15. The composition of claim 6, wherein the composition is loaded onto a patch for application to skin by:
loading the halloysite clay with green tea solubilized in water to form a clay-green tea slurry;
dissolving guar gum powder in water to form a 3% w/v concentration of guar gum;
adding preservative;
adding acrylic adhesive; and
loading the mixture of clay-green tea slurry, guar gum, preservative, and acrylic adhesive onto the patch.

16. The composition of claim 15, wherein the preservative comprises phenoxyethanol, and the acrylic adhesive comprises Loctite Duro-Tak129NA.

17. The composition of claim 6, wherein the clay carrier comprises one of halloysite clay and kaolin.

18. A composition for transdermal delivery, the composition comprising 40 to 60 percent natural gel wax base; about 2 to about 4 percent carrier oil, about 2 to about 4 percent surfactant; about 20 percent to about 30 percent by weight clay carrier; about 5 to about 15 percent by weight green tea; and about 1 to about 3 percent by weight preservative.

19. The composition of claim 18, wherein the natural gel wax base comprises at least one of castor seed oil, hydrogenated castor oil, beeswax, and carnuba wax.

20. The composition of claim 18, wherein the clay carrier comprises one of halloysite clay and kaolin.
